(19) United States
(12) Patent Application Publication
(10) Pub. No.: US 2022/0088121 A1
(43) Pub. Date: Mar. 24, 2022

(54) COMPOSITIONS CONTAINING A PEPTIDE ABLE TO STIMULATE THE GPRC6A-DEPENDENT SIGNALING PATHWAY

(71) Applicants: Altergon S.A., Lugano (CH); FONDAZIONE FORESTA - ONLUS, Padova (IT)

(72) Inventors: Carlo Foresta, Padova (IT); Luca De Toni, Padova (IT); Andrea Di Nisio, Padova (IT); Maurizio De Rocco Ponce, Padova (IT); Andrea Giori, Lugano (CH)

(73) Assignees: Altergon S.A., Lugano (CH); FONDAZIONE FORESTA - ONLUS, Padova (IT)

(21) Appl. No.: 17/421,477

(22) PCT Filed: Jan. 7, 2020
(86) PCT No.: PCT/IB2020/050074

§ 371 Date: Jul. 8, 2021

(30) Foreign Application Priority Data
Jan. 10, 2019 (IT) 102019000000367

(51) Int. Cl. A61K 38/16; A61P 5/26

(52) U.S. Cl. CPC A61K 38/168; A61P 5/26

(57) ABSTRACT

Disclosed are pharmaceutical or nutraceutical compositions comprising the peptide having the sequence NMYLPPVPPP PVVPTF or extracts containing it, in admixture with suitable excipients.

The present invention relates to pharmaceutical or nutraceutical compositions comprising the peptide having the sequence NMYLPPVPPP PVVPTF or extracts containing it, in admixture with suitable excipients. The compositions according to the invention are useful for therapeutic or adjuvant treatment of conditions that benefit from stimulation of the GPRC6A-dependent signalling pathway. Examples of such conditions include male hypogonadism, osteopenia/osteoporosis, erectile dysfunction and metabolic syndrome.

### BACKGROUND OF THE INVENTION

The role of bone tissue as an endocrine organ is a subject that has aroused great scientific interest in the last ten years. Initial studies of animal models suggested that osteocalcin (OCN), a small protein of osteoblastic origin which is normally involved in mineralisation of extracellular bone matrix in its γ-carboxylated form, may exhibit systemic effects after decarboxylation. In particular, uncarboxylated osteocalcin (ucOCN) appeared to be a direct stimulator of insulin release from the pancreatic islets, a protective factor in the maintenance of insulin sensitivity in the main target organs of the hormone, such as adipose tissue and muscle, and a regulator of bone mass and male fertility through control of the production and release of testosterone (T) by the Leydig cells in the testicles [Lee Cell. 2007; Ferron Proc Natl Acad Sci USA. 2008; Pi PLoS One. 2008; Rached J Clin Invest. 2010; Ferron Cell. 2010; Oury Cell. 2011]. In vitro and in vivo studies identified the GPRC6A receptor as the mediator of the systemic activity of ucOCN [Pi J Biol Chem. 2005; Pi J Bone Miner Res. 2011; Wei Diabetes. 2014]. Subsequent studies clarified that the endocrine role of ucOCN through the GPRC6A receptor was also preserved in humans, confirming the role of this novel multi-organ endocrine axis [Foresta J Clin Endocrinol Metab. 2010; Oury. J Clin Invest. 2013; De Toni Endocrinology. 2014; De Toni J Clin Endocrinol Metab. 2016; Di Nisio Clin Endocrinol (Oxf). 2017; Parker J Bone Miner Res. 2018].

The crucial characteristic of GPRC6A is that it is a metabotropic receptor, i.e. can be activated by a range of small molecules of metabolic interest such as basic amino acids (e.g. arginine and ornithine) and metal cations (e.g. calcium and magnesium) [Pi J Biol Chem. 2005]. In vitro studies conducted in the early 2010s suggested that GPRC6A could represent the T membrane receptor, i.e. the mediator of the “non-genomic” or rapid effects of androgens [Pi J Biol Chem. 2010; Pi Mol Endocrinol. 2015]. The correctness of this hypothesis was subsequently verified in numerous in vitro studies of cell lines and primary cell cultures characterised by constitutive expression of GPRC6A [Ko. J Biol Chem. 2014; Pi Mol Endocrinol. 2015; O'Hara FASEB J. 2015].

According to some authors, the hypothesis that GPRC6A represents the common molecular target of some highly chemically different entities such as amino acids, metal cations and steroids constitutes a biological model with some highly critical aspects [Clemmensen Br J Pharmacol. 2014]. In this respect, it should be borne in mind that the majority of T is present in the peripheral circulation as a complex with a serum globulin known as “sex-hormone-binding globulin” (SHBG) [Mean Clin Chim Acta. 1977]. Moreover, numerous studies have demonstrated a significant association between low plasma levels of SHBG and clinical signs of metabolic alterations such as increased insulin resistance and increased risk of type 2 diabetes [Birkeland J Clin Endocrinol Metab. 1993; Tsai Diabetes Care. 2004; Muller J Clin Endocrinol Metab. 2005; Pitteloud Diabetes Care. 2005; Ding N Engl J Med. 2009]. These factors, together with the fact that both ucOCN and SHBG are proteins, suggested that SHBG may act as a mediator in the interaction between T and GPRC6A. That hypothesis was confirmed in a recent study wherein, using an integrated computational-experimental approach, a common bonding site for interaction with both ucOCN and SHBG was identified in the extracellular domain of GPRC6A [De Toni Endocrinology 2016]. WO2009073544 proposes the possible therapeutic use of agents able to regulate the level and/or activity of GPRC6A, especially for the treatment of obesity and diabetes.

### DESCRIPTION OF THE INVENTION

It has now been discovered that an amino-acid sequence exhibits close homology (62.5% amino-acid identity; 75% structural similarity) with domain 141-161 of SHBG (SHBG₁₄₁₋₁₆₁), corresponding to a protein loop with high flexibility responsible for the interaction between ucOCN and GPRC6A.

It has also been found that said peptide sequence (NMYLPPVPPPPVVPTF, SEQ ID 1, P80762) stimulates insulin and testosterone release in experimental models of cell line cultures.

The object of the invention is therefore pharmaceutical or nutraceutical compositions comprising the peptide having the sequence NMYLPPVPPP PVVPTF in isolated form or isolated proteins containing said sequence or extracts containing said sequences or isolated proteins, in admixture with suitable excipients.

The compositions according to the invention are useful in the treatment of conditions that benefit from stimulation of the GPRC6A-dependent signalling pathway. Examples of such conditions include male hypogonadism, osteopenia/osteoporosis, erectile dysfunction and metabolic syndrome.

As well as the peptide having the sequence indicated, the invention also comprises the use of peptides that exhibit at least 75%, preferably at least 80%, and more preferably at least 90% homology, compared with sequence P80762 (SEQ ID 1). For example, peptides wherein sequence NMYLPPVPPPPVVPTF has been modified with conservative substitutions of amino acids and/or deletions of 1-4 amino acids can be used according to the invention.

The peptide NMYLPPVPPPPVVPTF and its homologues can be prepared by conventional peptide synthesis methods. Alternatively, a natural source containing said peptides, in particular Phaseolus vulgaris extracts containing the protein fraction of said species, can be used.

The extracts can be obtained from varieties of Phaseolus vulgaris subjected to suitable enzymatic hydrolysis processes followed by extraction with aqueous or alcohol-based solvents.

The compositions according to the invention can be administered orally, transdermally or parenterally.

Examples of suitable forms of administration include capsules, tablets, solutions, suspensions, gels, transdermal patches and the like. The peptides contained in the Phaseolus extract can be combined with other ingredients having complementary or otherwise useful activity such as vitamins, amino acids and antioxidants, minerals such as zinc and magnesium salts, and phytotherapeutic extracts (such as Panax ginseng, Ginkgo biloba, green tea, Vitis vinifera, Vaccinium myrtillus, Serenoa repens, Aloe vera, Cynara scolymus, Citrus aurantium and Zingiber officinalis).

The effective doses of the peptides will be determined by the skilled person on the basis of pre-clinical and clinical tests. The doses will depend on various factors, such as the type and severity of the patient's disorder or condition, and the patient's weight, sex and age. However, an average daily dose could range from 10 to 1000 mg of peptide, or the equivalent of the extract containing it.

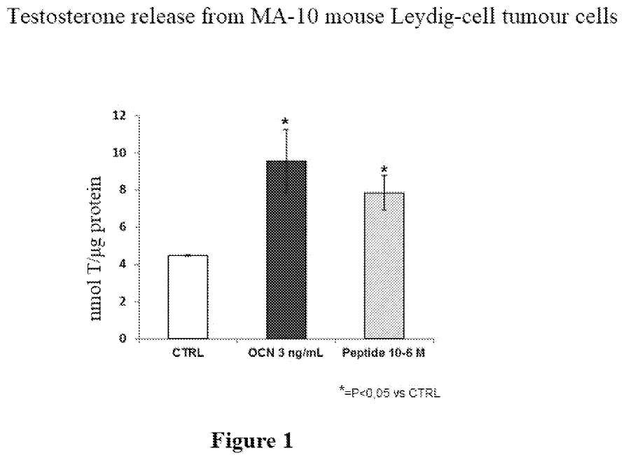

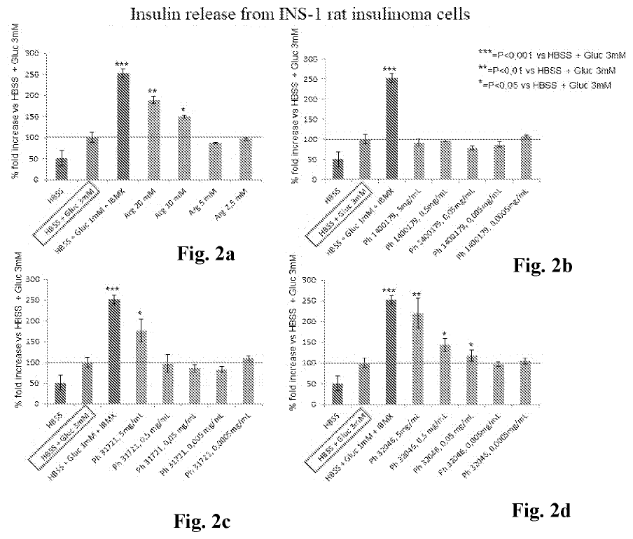

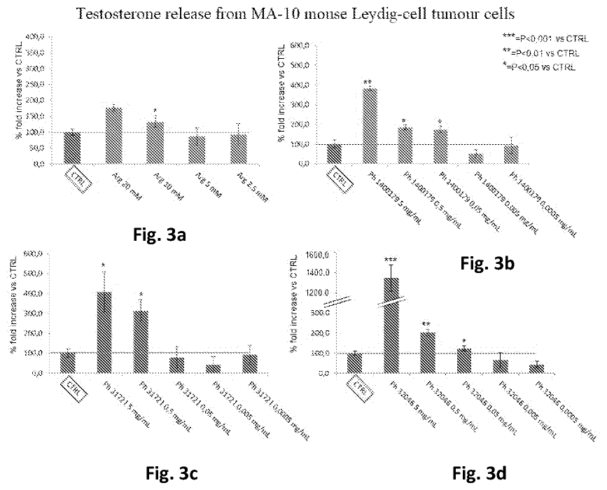

The efficacy of the formulations according to the invention can be deduced from in vitro studies conducted on INS-1 rat insulinoma and MA-10 mouse Leydig cell tumour cell lines, using kidney bean flour of commercial origin. The results, reported in the experimental section below, demonstrated the release of insulin (FIG. 2) and testosterone (FIGS. 1 and 3). Expression of GPRC6A was confirmed in both cell lines [Brar Diabetes. 2017; De Toni Endocrinology. 2014].

The following examples illustrate the invention in greater detail.

### Example 1 Testosterone Release from MA-10 Mouse Leydig-Cell Tumour Cell Cultures

MA-10 mouse Leydig-cell tumour cells, cultured to 90% confluence in 24-well multiwell plates, were stimulated with different agonists for 12 hours at 37° C. under sterile conditions, according to an already validated protocol [Cormier Cell Biol Toxicol. 2018]. The control (CTRL) contained no agonists. The results relate to testosterone release, expressed as ng of testosterone (T) per μg of total protein, obtained from cell extract. The histograms show the effect on testosterone release of uncarboxylated osteocalcin (OCN, 3 ng/mL) and peptide SEQ ID 1 (Peptide, 10⁻⁶M). Significance: *=P<0.05 vs. CTRL.

### Example 2 Insulin Release from INS-1 Murine Insulinoma Cell Cultures

INS-1 rat insulinoma cells, cultured to 90% confluence in 24-well multiwell plates, were stimulated with different combinations of secretagogue agonists for 2 hours at 37° C. under sterile conditions, according to an already validated protocol [Hohmeier Diabetes. 2000]. The results relate to insulin release, expressed as the normalised variation in low-concentration glucose (Gluc 3 mM) at the baseline control. Stimulation with the phosphodiesterase inhibitor IBMX (Gluc 1 mM+IBMX) represents the maximum secretagogue stimulation and positive control of the experiment. The four histograms in FIG. 2a show the effect on insulin release of arginine (Arg, at concentrations ranging from 2.5 to 20 mM), a known GPRC6A agonist, while the release of three Phaseolus extracts (Ph1400179, Ph31721 and Ph32046, at concentrations ranging from 0.0005 to 5 mg/mL), is shown in FIGS. 2b-d respectively. Significance: *, ** and ***=P<0.05, 0.01 and 0.001 respectively vs the baseline control.

### Example 3 Testosterone Release from MA-10 Mouse Leydig-Cell Tumour Cell Cultures

MA-10 mouse Leydig-cell tumour cells, cultured to 90% confluence in 24-well multiwell plates, were stimulated with different agonists for 12 hours at 37° C. under sterile conditions, according to an already validated protocol [Cormier Cell Biol Toxicol. 2018]. The control (CTRL) contained no agonists The results relate to testosterone release, expressed as normalised variation vs the CTRL. The four histograms in FIG. 3a show the effect on testosterone release of arginine (Arg, at concentrations ranging from 2.5 to 20 mM), a known GPRC6A agonist, while the release of three Phaseolus extracts (Ph1400179, Ph31721 and Ph32046, at concentrations ranging from 0.0005 to 5 mg/mL), is shown in FIGS. 3b-d respectively. Significance: *, ** and ***=P<0.05, 0.01 and 0.001 respectively vs the baseline control.

### REFERENCES

Birkeland K I, Hanssen K F, Torjesen P A, Vaaler S. Level of sex hormone binding globulin is positively correlated with insulin sensitivity in men with type 2 diabetes. J Clin Endocrinol Metab. 1993; 76(2):275-278.
Brar G S, Barrow B M, Watson M, Griesbach R, Choung E, Welch A, Ruzsicska B, Raleigh D P, Zraika S. Neprilysin Is Required for Angiotensin-(1-7)'s Ability to Enhance Insulin Secretion via Its Proteolytic Activity to Generate Angiotensin-(1-2). Diabetes. 2017 August; 66(8):2201-2212.
Clemmensen C, Smajilovic S, Wellendorph P, Bräuner-Osborne H. The GPCR, class C, group 6, subtype A (GPRC6A) receptor: from cloning to physiological function. Br J Pharmacol. 2014 March; 171(5):1129-41. doi: 10.1111/bph.12365.
Cormier M, Ghouili F, Roumaud P, Bauer W, Touaibia M, Martin L J. Influences of flavones on cell viability and cAMP-dependent steroidogenic gene regulation in M A-10 Leydig cells. Cell Biol Toxicol. 2018 February; 34(1):23-38.
De Toni L, De Filippis V, Tescari S, Ferigo M, Ferlin A, Scattolini V, Avogaro A, Vettor R, Foresta C. Uncarboxylated osteocalcin stimulates 25-hydroxy vitamin D production in Leydig cell line through a GPRC6a-dependent pathway. Endocrinology. 2014 November; 155(11):4266-74.
De Toni L, Di Nisio A, Speltra E, Rocca M S, Ghezzi M, Zuccarello D, Turiaco N, Ferlin A, Foresta C. Polymorphism rs2274911 of GPRC6A as a Novel Risk Factor for Testis Failure. J Clin Endocrinol Metab. 2016 March; 101(3):953-61.
De Toni L, Guidolin D, De Filippis V, Tescari S, Strapazzon G, Santa Rocca M, Ferlin A, Plebani M, Foresta C. Osteocalcin and Sex Hormone Binding Globulin Compete on a Specific Binding Site of GPRC6A. Endocrinology. 2016 November; 157(11):4473-4486.
Di Nisio A, Rocca M S, Fadini G P, De Toni L, Marcuzzo G, Marescotti M C, Sanna M, Plebani M, Vettor R, Avogaro A, Foresta C. The rs2274911 polymorphism in GPRC6A gene is associated with insulin resistance in normal weight and obese subjects. Clin Endocrinol (Oxf). 2017 February; 86(2):185-191.
Ding E L, Song Y, Manson J E, Hunter D J, Lee C C, Rifai N, Buring J E, Gaziano J M, Liu S. Sex hormone-binding globulin and risk of type 2 diabetes in women and men. N Engl J Med. 2009 Sep. 17; 361(12):1152-63.
Ferron M, Hinoi E, Karsenty G, Ducy P. Osteocalcin differentially regulates beta cell and adipocyte gene expression and affects the development of metabolic diseases in wild-type mice. Proc Natl Acad Sci USA. 2008 Apr. 1; 105(13):5266-70.
Ferron M, Wei J, Yoshizawa T, Del Fattore A, DePinho R A, Teti A, Ducy P, Karsenty G. Insulin signaling in osteoblasts integrates bone remodeling and energy metabolism. Cell. 2010 Jul. 23; 142(2):296-308.
Foresta C, Strapazzon G, De Toni L, Gianesello L, Calcagno A, Pilon C, Plebani M, Vettor R. Evidence for osteocalcin production by adipose tissue and its role in human metabolism. J Clin Endocrinol Metab. 2010 July; 95(7):3502-6.
Hohmeier H E, Mulder H, Chen G, Henkel-Rieger R, Prentki M, Newgard C B. Isolation of INS-1-derived cell lines with robust ATP-sensitive K+ channel-dependent and -independent glucose-stimulated insulin secretion. Diabetes. 2000 March; 49(3):424-30.
Ko E, Choi H, Kim B, Kim M, Park K N, Bae I H, Sung Y K, Lee T R, Shin D W, Bae Y S. Testosterone stimulates Duox1 activity through GPRC6A in skin keratinocytes. J Biol Chem. 2014 Oct. 17; 289(42):28835-45.
Lee N K, Sowa H, Hinoi E, Ferron M, Ahn J D, Confavreux C, Dacquin R, Mee P J, McKee M D, Jung D Y, Zhang Z, Kim J K, Mauvais-Jarvis F, Ducy P, Karsenty G. Endocrine regulation of energy metabolism by the skeleton. Cell. 2007 Aug. 10; 130(3):456-69.
Mean F, Pellaton M, Magrini G. Study on the binding of dihydrotestosterone, testosterone and oestradiol with sex hormone binding globulin. Clin Chim Acta. 1977; 80(1):171-180.
Muller M, Grobbee D E, den Tonkelaar I, Lamberts S W, van der Schouw Y T. Endogenous sex hormones and metabolic syndrome in aging men. J Clin Endocrinol Metab. 2005; 90(5):2618-2623.
O'Hara L, McInnes K, Simitsidellis I, Morgan S, Atanassova N, Slowikowska-Hilczer J, Kula K, Szarras-Czapnik M, Milne L, Mitchell R T, Smith L B. Autocrine androgen action is essential for Leydig cell maturation and function, and protects against late-onset Leydig cell apoptosis in both mice and men. FASEB J. 2015 March; 29(3):894-910.
Oury F, Ferron M, Huizhen W, Confavreux C, Xu L, Lacombe J, Srinivas P, Chamouni A, Lugani F, Lejeune H, Kumar T R, Plotton I, Karsenty G. Osteocalcin regulates murine and human fertility through a pancreas-bone-testis axis. J Clin Invest. 2013 June; 123(6):2421-33.
Oury F, Sumara G, Sumara O, Ferron M, Chang H, Smith C E, Hermo L, Suarez S, Roth B L, Ducy P, Karsenty G. Endocrine regulation of male fertility by the skeleton. Cell. 2011 Mar. 4; 144(5):796-809.
Parker L, Lin X, Garnham A, McConell G, Stepto N K, Hare D L, Byrnes E, Ebeling P R, Seeman E, Brennan-Speranza T C, Levinger I. Glucocorticoid-Induced Insulin Resistance in Men Is Associated With Suppressed Undercarboxylated Osteocalcin. J Bone Miner Res. 2018 Aug. 23. doi: 10.1002/jbmr.3574.
Pi M, Chen L, Huang M Z, Zhu W, Ringhofer B, Luo J, Christenson L, Li B, Zhang J, Jackson P D, Faber P, Brunden K R, Harrington J J, Quarles L D. GPRC6A null mice exhibit osteopenia, feminization and metabolic syndrome. PLoS One. 2008; 3(12): e3858.
Pi M, Faber P, Ekema G, Jackson P D, Ting A, Wang N, Fontilla-Poole M, Mays R W, Brunden K R, Harrington J J, Quarles L D. Identification of a novel extracellular cation-sensing G-protein-coupled receptor. J Biol Chem. 2005 Dec. 2; 280(48):40201-9.
Pi M, Kapoor K, Wu Y, Ye R, Senogles S E, Nishimoto S K, Hwang D J, Miller D D, Narayanan R, Smith J C, Baudry J, Quarles L D. Structural and Functional Evidence for Testosterone Activation of GPRC6A in Peripheral Tissues. Mol Endocrinol. 2015 December; 29(12):1759-73.
Pi M, Parrill A L, Quarles L D. GPRC6A mediates the non-genomic effects of steroids. J Biol Chem. 2010 Dec. 17; 285(51):39953-64.
Pi M, Wu Y, Quarles L D. GPRC6A mediates responses to osteocalcin in β-cells in vitro and pancreas in vivo. J Bone Miner Res. 2011 July; 26(7):1680-3.
Pitteloud N, Mootha V K, Dwyer A A, et al. Relationship between testosterone levels, insulin sensitivity, and mitochondrial function in men. Diabetes Care. 2005; 28(7): 1636-1642.
Rached M T, Kode A, Silva B C, Jung D Y, Gray S, Ong H, Paik J H, DePinho R A, Kim J K, Karsenty G, Kousteni S. FoxO1 expression in osteoblasts regulates glucose homeostasis through regulation of osteocalcin in mice. J Clin Invest. 2010 January; 120(1):357-68.
Tsai E C, Matsumoto A M, Fujimoto W Y, Boyko E J. Association of bioavailable, free, and total testosterone with insulin resistance: influence of sex hormone-binding globulin and body fat. Diabetes Care. 2004; 27(4):861-868.
Wei J, Hanna T, Suda N, Karsenty G, Ducy P. Osteocalcin promotes β-cell proliferation during development and adulthood through Gprc6a. Diabetes. 2014 March; 63(3): 1021-31.

1. Pharmaceutical or nutraceutical compositions comprising a peptide having sequence NMYLPPVPPPPVVPTF (SEQ ID NO:1) in isolated form or isolated proteins containing said sequence or extracts containing said sequences or isolated proteins.

2. Compositions according to claim 1 wherein the peptide is contained in extracts of Phaseolus vulgaris.

3. Compositions according to claim 1 further comprising additional active ingredients having complementary or synergic activity.

4. Method of treating conditions benefiting from stimulation of the GPRC6A-dependent signalling pathway with the compositions according to claim 1 in patients in need thereof, said method comprising
administering said compositions to said patient.

5. The method according to claim 4 wherein the conditions comprise male hypogonadism, osteopenia/osteoporosis, erectile dysfunction and metabolic syndrome.


SEQUENCE LISTING (1 sequence)

SEQ ID NO: 1 - protein, 16 aa, Artificial Sequence
  Synthetic peptide
NMYLPPVPPPPVVPTF